(19) United States
(12) Patent Application Publication
(10) Pub. No.: US 2016/0334385 A1
(43) Pub. Date: Nov. 17, 2016

(54) METHODS AND APPARATUS FOR REPRESENTING BLOOD GLUCOSE VARIATION GRAPHICALLY

(71) Applicant: Ascensia Diabetes Care Holdings AG, Basel (CH)

(72) Inventors: Eugene Prais, West Milford, NJ (US); Robert W. Morin, Boca Raton, FL (US)

(21) Appl. No.: 15/110,739

(22) PCT Filed: Dec. 31, 2014
(86) PCT No.: PCT/US14/72909

§ 371 Date: Jul. 9, 2016

Related U.S. Application Data
(60) Provisional application No. 61/926,210, filed on Jan. 10, 2014.

(51) Int. Cl. G01N 33/49; G01N 33/487; G06T 11/20

(52) U.S. Cl. CPC G01N 33/49; G06T 11/206; G01N 33/48792

(57) ABSTRACT

Graphical representation of glycemic variation and average blood glucose values. The invention comprises storing in a memory a series of blood glucose values measured over a period of time; determining, using a processor, a variation value for the series of blood glucose values during the time period based on a standard deviation of the series of blood glucose values; plotting points using the processor for each of the blood glucose values around a center point where the radial distance from the center point to each point to be plotted is determined based on the variation value and a blood glucose value of the point to be plotted, and outputting to a display operative coupled to the processor, a graphical representation of the plotted series of blood glucose values and an indicia representative of the variation value.

### RELATED APPLICATIONS

The present application claims priority to U.S. Provisional Patent Application Ser. No. 61/926,210, filed Jan. 10, 2014 and entitled “METHODS AND APPARATUS FOR REPRESENTING BLOOD GLUCOSE VARIATION GRAPHICALLY”, (Attorney Docket No. BHC134021(BHDD/044/L)), which is hereby incorporated herein by reference in its entirety for all purposes.

### FIELD

The present invention relates to medical devices, and more specifically to apparatus, systems, and methods for graphical representation of blood glucose variation.

### BACKGROUND

Conventional end user medical devices such as blood glucose meters (BGMs) typically display actual blood glucose (BG) levels and an average BG level maintained over a period of time. Some displays provide timeline plots of the BG data in an effort to indicate when the user's BG levels are out of the desired range. Unfortunately however, such displays do not provide the user with a tangible understanding of the effectiveness of the user's efforts to control his BG levels. Accordingly, systems, apparatus, and methods for providing a graphical representation of a user's blood glucose variation are needed.

### SUMMARY

In some aspects, embodiments of the present invention provide an apparatus for displaying a graphical representation of a user's blood glucose variation. The apparatus includes a processor; a display operatively coupled to the processor; and a memory operatively coupled to the processor and operative to store processor executable instructions adapted to store in the memory a series of blood glucose values measured over a period of time, determine using the processor a variation value for the series of blood glucose values during the time period based on a standard deviation of the series of blood glucose values, plot points using the processor for each of the blood glucose values around a center point where the radial distance from the center point to each point to be plotted is determined based on the variation value and a blood glucose value of the point to be plotted, and output to the display a graphical representation of the plotted series of blood glucose values and an indicia representative of the variation value. In some embodiments, the apparatus can be embodied as a smart phone, personal digital assistant, tablet, personal computer, mobile device, cell phone, laptop, blood glucose monitor, or other device.

In other aspects, embodiments of the present invention provide a system for providing a graphical representation of a user's blood glucose variation. The system includes a processor; a port operatively coupled to the processor and adapted to receive a blood glucose test strip; a display operatively coupled to the processor; and a memory operatively coupled to the processor and operative to store processor executable instructions adapted to measure blood glucose levels in blood samples applied to test strips received in the port and to determine blood glucose values from the measurements, store in the memory at least two series of blood glucose values measured over at least two different periods of time, determine using the processor a variation value for each of the series of blood glucose values during the respective time periods based on a standard deviation of each of the series of blood glucose values, plot points using the processor for a first series for each of the blood glucose values around a first center point where the radial distance from the first center point to each point of the first series to be plotted is determined based on the variation value of the first series and a blood glucose value of the point to be plotted, plot points using the processor for a second series for each of the blood glucose values around a second center point where the radial distance from the second center point to each point of the second series to be plotted is determined based on the variation value of the second series and a blood glucose value of the point to be plotted, and output to the display a graphical representation of both the first and second plotted series of blood glucose values and an indicia representative of the variation values of the first and second series.

In yet other aspects, embodiments of the present invention provide a method for providing a graphical representation of a user's blood glucose variation. The method includes storing in a memory, a series of blood glucose values measured over a period of time; determining, using a processor operatively coupled to the memory, a variation value for the series of blood glucose values during the time period based on a standard deviation of the series of blood glucose values; plotting points using the processor for each of the blood glucose values around a center point where the radial distance from the center point to each point to be plotted is determined based on the variation value and a blood glucose value of the point to be plotted, and outputting to a display operative coupled to the processor, a graphical representation of the plotted series of blood glucose values and an indicia representative of the variation value.

Numerous other aspects are provided in accordance with these and other embodiments of the invention. Other features and aspects of embodiments of the present invention will become more fully apparent from the following detailed description, the appended claims, and the accompanying drawings.

### BRIEF DESCRIPTION OF THE DRAWINGS

FIG. 1 illustrates an example system including a first example graphical display of BG variation according to some embodiments of the present invention.

FIG. 2 illustrates a second example graphical display of BG variation according to some embodiments of the present invention.

FIG. 3 illustrates a third example graphical display of BG variation according to some embodiments of the present invention.

FIG. 4 illustrates a fourth example graphical display of BG variation according to some embodiments of the present invention.

FIG. 5 illustrates a fifth example graphical display of BG variation according to some embodiments of the present invention.

FIG. 6 illustrates a sixth example graphical display of BG variation according to some embodiments of the present invention.

FIG. 7 illustrates a seventh example graphical display of BG variation according to some embodiments of the present invention.

FIG. 8 illustrates an eighth example graphical display of BG variation according to some embodiments of the present invention.

FIG. 9 is a flowchart depicting a method of displaying a graphical representation of BG variation according to some embodiments of the present invention.

### DETAILED DESCRIPTION

The detection of the concentration level of glucose or other analyte in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

End user analyte monitoring and measurement medical devices such as, for example, blood glucose meters (BGMs) are typically provided to patients by a healthcare provider (HCP). Some BGMs are adapted to receive a test strip that allows a blood sample to be tested while other BGMs are adapted to receive a signal from a transdermal in vivo sensor worn by the user. In either case the BGMs typically include a processor and memory that are used to store the BG data and a display to output information based on the BG data to the user. In addition, many BGMs further include communication facilities, (e.g., wireless transceivers, I/O ports, etc.) that facilitate transfer of the BG data to a computer, a smart phone, a tablet, or other display device, to allow the information to be displayed to the user or HCP in various different forms.

An important purpose of displaying the information based on the BG data is to help the user/patient and the HCP better understand the user's level of control over his BG level. Based on this understanding, the user can take steps to improve or maintain his control over his BG levels and the HCP can prescribe medication and regimens (e.g., dietary, exercise, etc.) for the same purpose.

Tighter glycemic control is a key factor in treatment of diabetes. Short term wide swings in BG levels carry with them detrimental effects that accumulate over time. As noted above however, conventional BGMs and other BG data display systems typically only depict plots of BG values and the average BG value. Since short term wide swings are hidden by averaging the values and mere plots of BG values on a time scale do not provide a clear indication of the significance or weight of values that fall outside the target ranges, such displays alone are not optimal indicators of a user's glycemic control. Embodiments of the present invention provide methods of quantifying glycemic variation over a given period of time using a tangible and intuitive graphic representation. Embodiments further provide representations that facilitate comparison and contrast of glycemic variation with that of other time periods to concurrently provide both BG averages and variation target ranges. Thus, embodiments of the present invention provide an effective means to represent and communicate glycemic variation on a BGM display, a smartphone application, or any other patient/HCP interface.

Conventionally, standard deviation is used as the standard measure of variation. In statistics and probability theory, the standard deviation (represented by the Greek letter sigma, σ) shows how much variation or dispersion from the average exists. A low standard deviation indicates that the data points tend to be very close to the mean (also called expected value); a high standard deviation indicates that the data points are spread out over a large range of values. For a finite set of numbers, the standard deviation is found by taking the square root of the average of the squared differences of the values from their average value. While familiar and useful to many trained HCPs, the concept of standard deviation is relatively abstract and technical for patients to easily understand and use. Thus, embodiments of the present invention represent standard deviation graphically, for example, as a non-linearly scaled unit-less value called “glycemic variation” or simply “variation.” Therefore, embodiments of the present invention make it unnecessary for patients to understand the standard deviation calculation and concept to be able to visualize and use an indication of variation in the therapeutic management of BG levels.

Clinically derived, medical based guidelines for medically acceptable standard deviation targets are known in the art. For example, the following equation expresses a relationship between standard deviation and average BG values that represents a recommended level of glucose variability.

2σ<BG_{avg}

where σ represents standard deviation and BG_{avg} represents the average BG value. Current research is actively quantifying recommended values for acceptable standard deviation for various types of patients with different conditions. Embodiments of the present invention can use any such values as a parameter in deriving the variation representation.

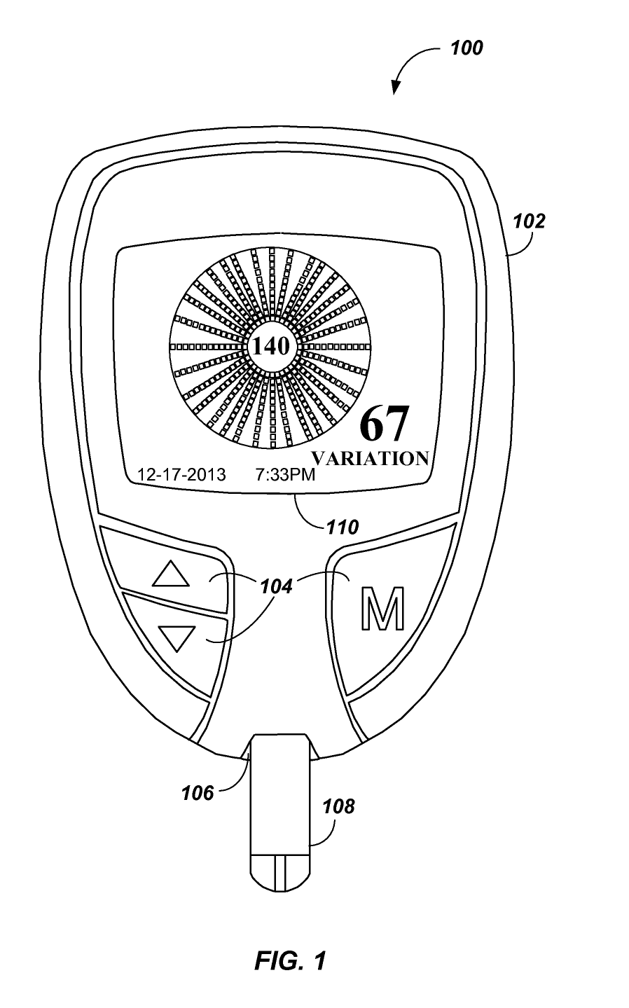

Turning now to FIG. 1, an example of a blood glucose meter 100 according to embodiments of the present invention is depicted. The BGM 100 depicted includes a housing 102 that stores and protects a circuit board (not visible) which includes a processor and memory. The memory is operative to store instructions executable on the processor as well as data such a BG data. The example BGM 100 shown further includes various user controls 104 that are operatively coupled to the processor for controlling operation of some of the functions of the BGM 100. The particular example BGM 100 shown also includes a port 106 for receiving a test strip 108. The test strip 108 includes a sensor adapted to receive a blood sample and to allow the processor within the BGM 100 to determine the glucose concentration of the blood. Note that in other embodiments, instead of or in addition to the port 106, the BGM 100 can include a wireless transceiver to enable receipt of a BG data signal transmitted from an in vivo sensor worn by the patient. Finally, the example BGM 100 includes a display 110, also coupled to the processor and adapted to output a representation to the user that concurrently conveys both average BG and variation information. In some embodiments, smartphones, tablets, or other devices can be used to store instructions and data, execute instructions, and display data and other representations.

The display 110 of the BGM 100 of FIG. 1 includes a first example representation with a simple starburst pattern that represents the standard deviation (SD) value as the variation. The radius of the pattern of this basic representation is a constant value that is presented to the user to graphically convey how much glycemic control they are maintaining over a period of time. A numerical representation of variation is displayed as well as a numerical representation of the average BG value.

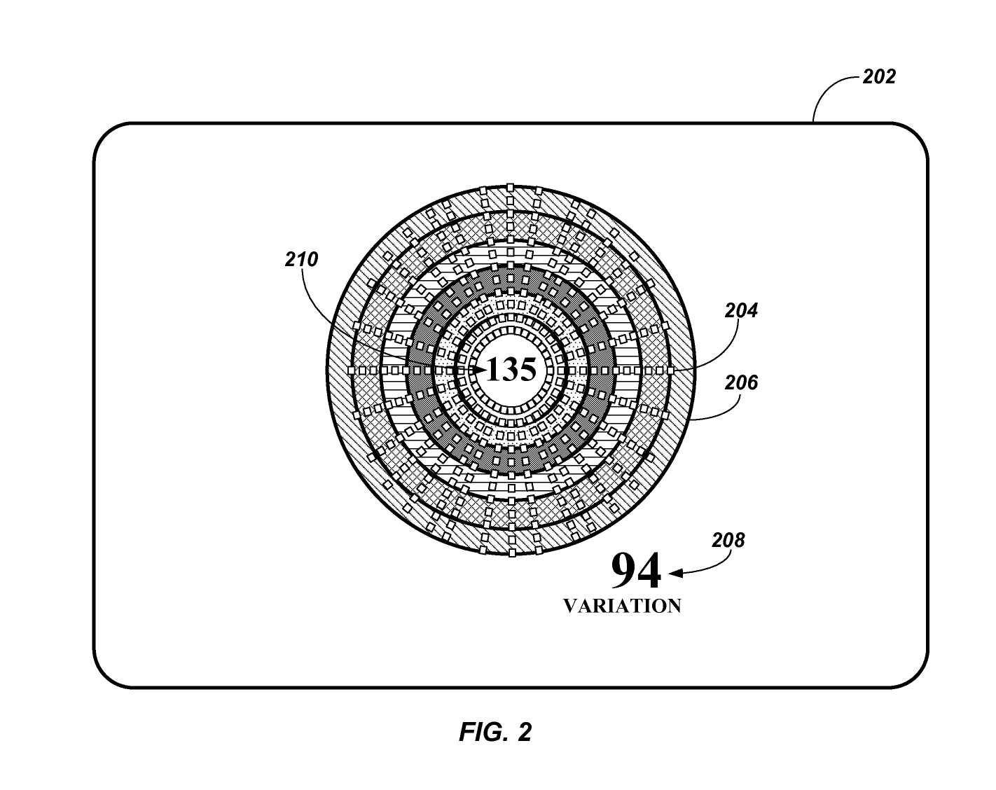

FIG. 2 depicts an example display 202 that includes a second example graphical representation of variation as a starburst pattern 204 of radially plotted BG values overlaid on a multi-zone colored target 206. A numerical representation of variation 208 is displayed as well as a numerical representation of the average BG value 210.

In some embodiments, the starburst pattern 204 of radially plotted BG values includes symbols (e.g., squares) each disposed at a distance from a center point that is determined based upon a scaled value selected to collectively create a visual representation of overall variation. For example, a scaling factor (e.g., 0.05) can be selected based on the available display area (e.g., 40 mm) and a min/max range of BG values (e.g., 0 to 400 mg/dl). The scaling factor can be used to convert BG values to radial plot distances. In some alternative embodiments, the scaling factor can be set to unity (or not used at all) and the BG values can be plotted directly as radially distance values.

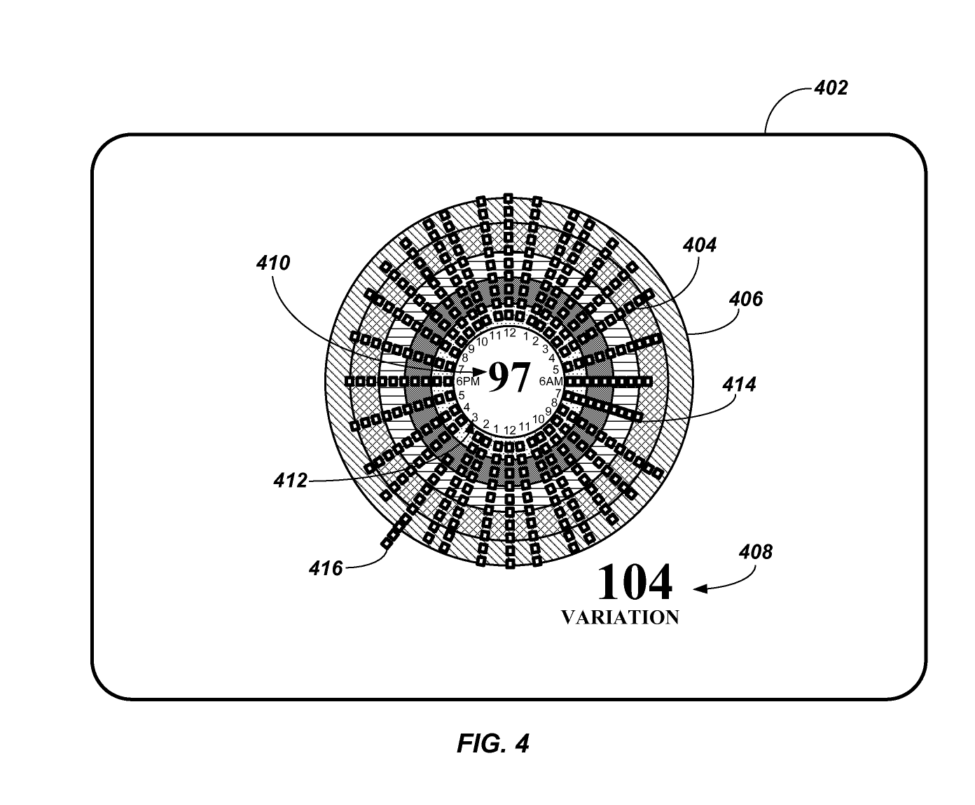

Note that in the example embodiment depicted, the angular position of the symbols does not represent any particular information. However, as will be described below with respect to FIG. 4, in alternative embodiments, the relative angular position of the symbols can represent time of day of the data point, date of the data point, etc.

In some embodiments, other symbols can be used such as circles, dots, triangles, hexagons, etc. In some embodiments, the symbols used can represent information about the particular data point. For example, squares can be used for BG values measured before meals and triangle can be used for BG values measured after meals. In some embodiments, shapes can be used along with radial position to indicate BG values.

In some embodiments, the width of the zones of the target 206 can be selected to be relatively proportionate to a percentage of the standard deviation. For example, the width of each zone can be set to 0.5σ multiplied by the scaling factor. Thus, the zones can provide a scale to gauge the amount of variation in the plotted BG values.

In some embodiments, the zones of the target 206 can be colored differently as indicated by the different fill patterns. In some embodiments, the colors can be selected to emphasize the level or severity of variation being represented. For example, the outermost zones can be colored bright “hot” (longer wavelength) colors such red, orange, and yellow while the innermost zones can be colored darker “cool” (shorter wavelength) colors such as violet, blue and green.

The numerical representation of variation 208 can be calculated as a function of standard deviation of the BG values collected over the time period of the BG data. In some embodiments, the numerical representation of variation 208 can be calculated by scaling the standard deviation to a particular range. For example, given sample glucose data of Blood Glucose in mg/dl: 84, 129, 202, 168, and 119, the variation would be calculated by determining the Mean as 140.4 and the SD as 45.62 using standard equations. Since 2×SD is ideally less than the Mean, this value of variability as expressed by the SD is acceptable, and the blood sugar values represent a reasonable level of fluctuation.

In some embodiments, instead of overlaying the starburst pattern 204 on target 206, the symbols of the starburst pattern 204 can be color-coded based on their radial distance from the center point. Thus, for example, data points relatively far from the center can be colored bright red, data points falling relatively close to the center can be colored dark blue/violet, and points in between can be colored based upon corresponding spectrum colors (e.g., the shorter the radial distance, the shorter the wavelength color used).

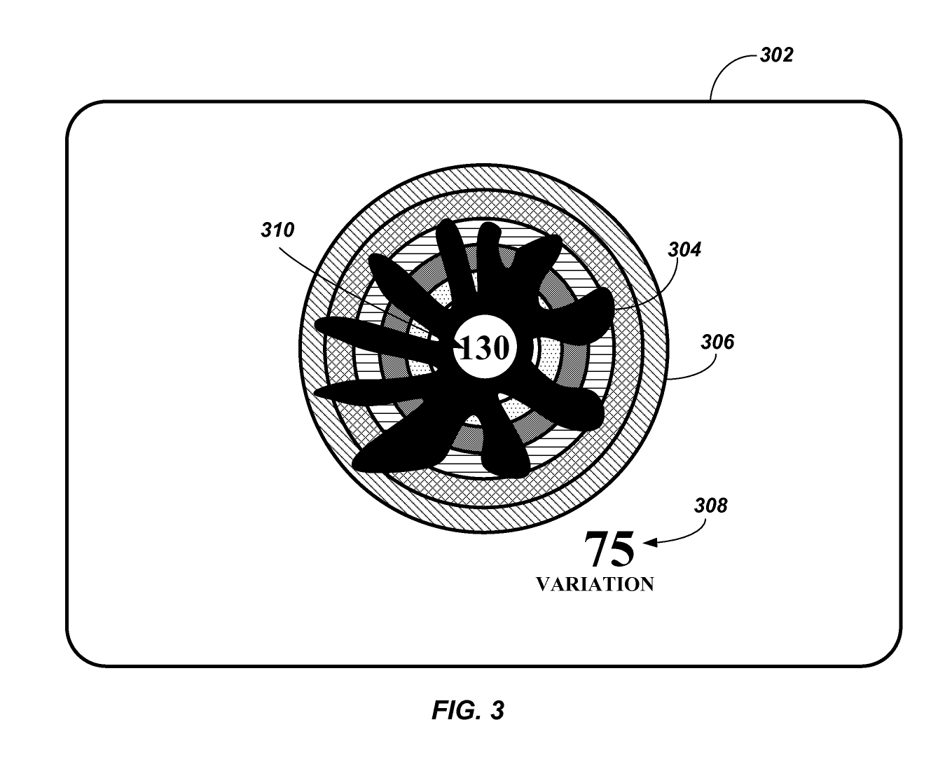

FIG. 3 depicts a display 302 with a third example graphical representation of variation as a “splatter” pattern 304 of aggregated BG values overlaid on a multi-zone colored target 306. As with the first example, a numerical representation of variation 308 is displayed as well as a numerical representation of the average BG value 310. In some embodiments, the splatter pattern 304 can be determined based upon scaled BG values plotted proportionate distances from the center point (e.g., using an appropriately selected scaling factor as described above) and then connected by filling in a trace of an outline of the outermost points.

As with the second example representation, the width of the zones of the target 306 can be selected to be relatively proportionate to a percentage of the standard deviation. For example, the width of each zone can be set to 0.5σ multiplied by the scaling factor. Thus, the zones can provide a scale to gauge the amount of variation in the plotted BG values.

Further, the zones of the target 306 can be colored differently as indicated by the different fill patterns. In some embodiments, the colors can be selected to emphasize the level or severity of variation being represented. For example, the outermost zones can be colored bright “hot” (longer wavelength) colors such red, orange, and yellow while the innermost zones can be colored darker “cool” (shorter wavelength) colors such as violet, blue and green.

In some embodiments, instead of overlaying the splatter pattern 304 on target 306, the “fingers” of the splatter pattern 304 can be color-coded based on their radial distance from the center point. Thus, for example, portions relatively far from the center can be colored bright red, portions relatively close to the center can be colored dark blue/violet, and portions in between can be colored based upon corresponding spectrum colors (e.g., the shorter the radial distance, the shorter the wavelength color used).

Turning to FIG. 4, a display 402 is depicted with a fourth example graphical representation of variation as a starburst pattern 404 of radially plotted BG values overlaid on a multi-zone colored target 406. A numerical representation of variation 408 is displayed as well as a numerical representation of the average BG value 410. As with the example shown in FIG. 2, the starburst pattern 404 of radially plotted BG values includes symbols (e.g., squares) each disposed at a distance from a center point that is determined based upon a scaled value selected to collectively create a visual representation of overall variation. For example, a scaling factor (e.g., 0.05) can be selected based on the available display area (e.g., 40 mm) and a min/max range of BG values (e.g., 0 to 400 mg/dl). The scaling factor can be used to convert BG values to radial plot distances. In some alternative embodiments, the scaling factor can be set to unity (or not used at all) and the BG values can be plotted directly as radially distance values.

The embodiment of FIG. 4 also includes a modal time of day indication 412 which indicates the time at which the data points plotted at the corresponding angular positions were measured. Thus, for example, each of the points plotted in the line of points extending from “7” and labeled with reference numeral 414 were measured at approximately 7 AM on different days within the monitoring period. Note that these data points 414 are relatively close to the center and compact in their distribution. In contrast, the data points 416 that were each measured at approximately 3 PM are relatively far from the center and spread out in their distribution. This example data suggests that the patient is repeatedly engaging in some activity (e.g., eating a candy bar) at approximately 3 PM each day that is causing his BG levels to rise significantly. Further, the data points 414 at approximately 7 AM suggest that the patient's “fasting” BG levels are relatively low but shortly after the day starts, the BG levels rise dramatically.

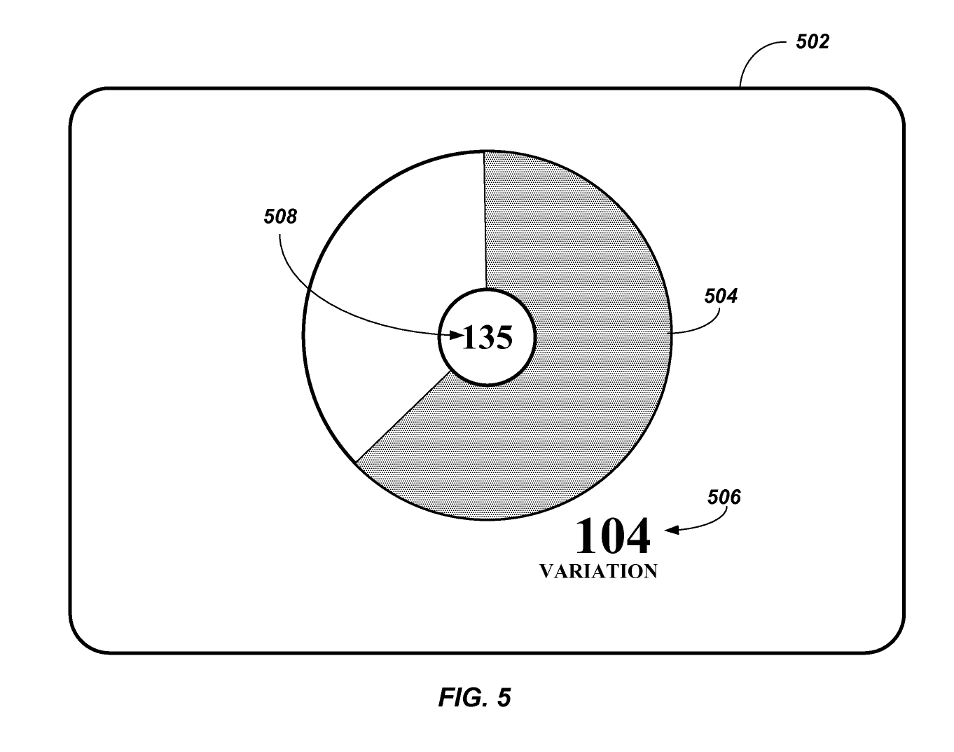

FIG. 5 depicts a display 502 with a fifth example graphical representation of variation and average BG level. In FIG. 5, the filled portion 504 provides an indication of variation. For example, the standard deviation can be scaled to be a percentage of 360 degrees and the filled portion 504 can be filled to the corresponding percentage. Thus, the larger the standard deviation, the high the percentage of the filled portion 504. In the example depicted, approximately 66% of the graphic is filled. As with the prior example embodiments, a numerical representation of variation 506 is displayed as well as a numerical representation of the average BG value 508.

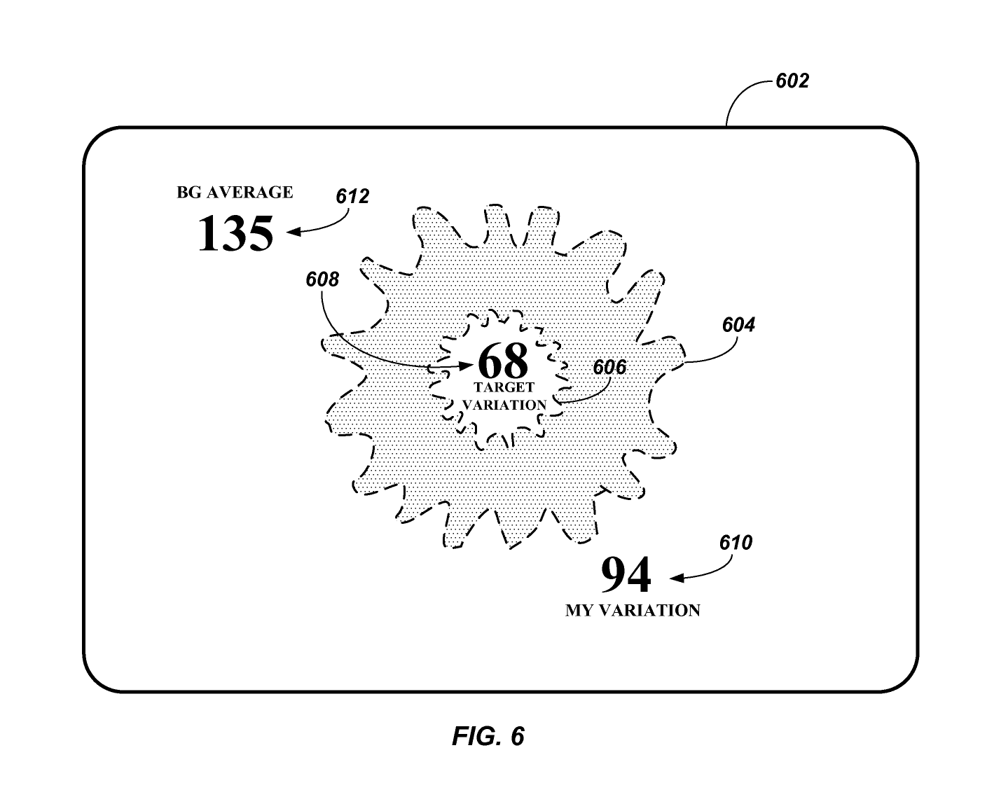

FIG. 6 depicts a display 602 with a sixth example graphical representation of variation and average BG level for a given period. In this example, a user “splatter” graphic 604 representing the user's glycemic variation is overlaid on a target “splatter” graphic 606 representing a medically acceptable variation that the user can use as a goal to reduce his variation and improve his glycemic control. As with the prior examples, the relative sizes of the graphics 604, 606 are scaled to provide the user with a proportionate indication of variation. Within the target splatter graphic 606, a numerical representation of the target variation value 608 can be displayed as shown. As with the prior example embodiments, a numerical representation of the user's variation 610 is displayed as well as a numerical representation of the average BG value 612.

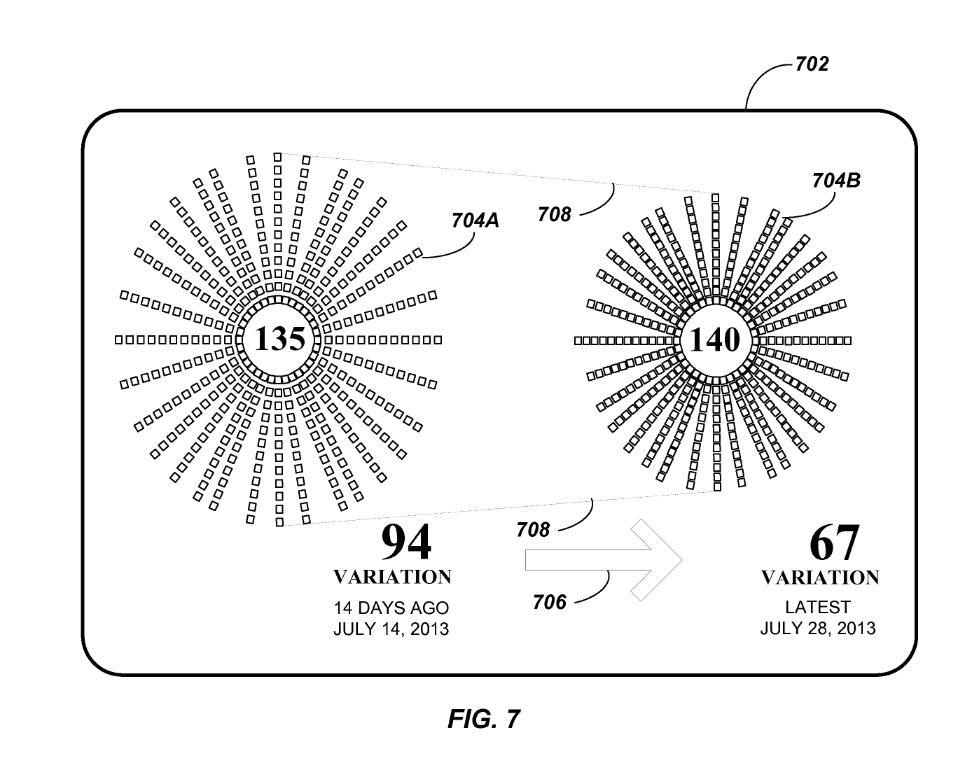

FIG. 7 depicts a display 702 with a seventh example graphical representation of variation and average BG level. In this example, two different graphics that each include starburst patterns 704A, 704B are displayed side by side for comparison. Each pattern 704A, 704B represents a different time period of BG monitoring and the arrow 706 indicates the passage of time. For example, the left side pattern 704A represents BG values measured over a two week period that ended “14 days ago, Jul. 14, 2013” and the right side pattern 704B represents the “latest” BG values measured over a two week period that ended “Jul. 28, 2013.” Note that even though the user's average BG level increased from 135 mg/dl to 140 mg/dl the variation decreased from 94 to 67. This positive change is further indicated by the tighter grouping of plotted values visible in the right side pattern 704B compared to the left side pattern 704A. In addition, the dotted lines 708 connecting the two patterns 704A, 704B further highlight the reduced glycemic variation.

In some embodiments, in order to further emphasize and illustrate the change in variation, an “emphasis factor” can be used. An emphasis factor is a constant (e.g., 0.8) that is multiplied by the BG values of the pattern with the smaller variation to make the visual difference between the two patterns to appear larger. In some embodiments, the emphasis factor used can be varied based on the actual difference between the two variations. For example, the smaller the variation difference, a lower value can be used for the emphasis factor to increase the relative sizes of the starburst patterns 704A, 704B. In some embodiments, the spacing between the two patterns 704A, 704B can be used to indicate the amount of time that has passed between the monitoring periods.

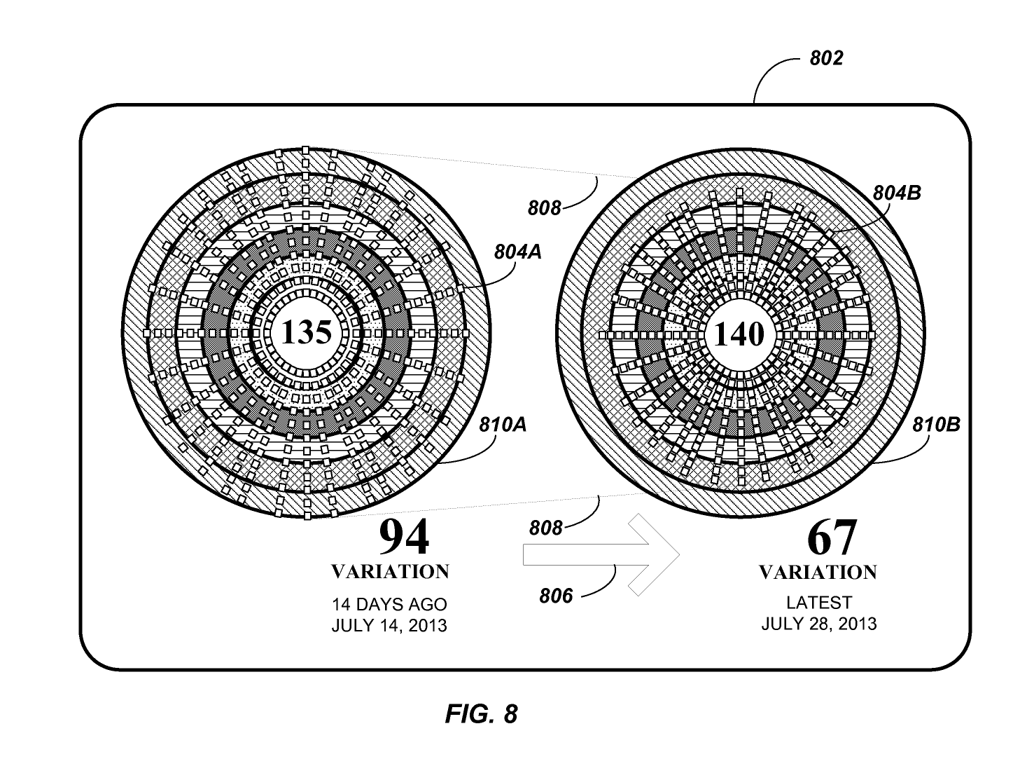

FIG. 8 depicts a display 802 with an eighth example graphical representation of variation and average BG level. As in the last example, two different graphics that each include starburst patterns 804A, 804B are displayed side by side for comparison and overlaid on multi-zoned color targets 810A, 810B, respectively. Each pattern 804A, 804B represents a different time period of BG monitoring and the arrow 806 indicates the passage of time. For example, the left side pattern 804A represents BG values measured over a two week period that ended “14 days ago, Jul. 14, 2013” and the right side pattern 804B represents the “latest” BG values measured over a two week period that ended “Jul. 28, 2013.” Note that even though the user's average BG level increased from 135 mg/dl to 140 mg/dl the variation decreased from 94 to 67. This positive change is further indicated by the tighter grouping of plotted values visible in the right side pattern 804B compared to the left side pattern 804A. In addition, the dotted lines 808 connecting the two patterns 804A, 804B further highlight the reduced glycemic variation. The targets 810A, 810B also provide an aid in comparing the two patterns 804A, 804B. As discussed above with respect to the other embodiments, similar targets 810A, 810B can include colors and dimensions to provide an enhanced understanding of the relative variation.

As discussed above in some embodiments, in order to further emphasize and illustrate the change in variation, an “emphasis factor” can be used. The emphasis factor can be a constant or can be varied based on the actual difference between the two variations. For example, the smaller the variation difference, a lower value can be used for the emphasis factor to increase the relative sizes of the starburst patterns 804A, 804B. In some embodiments, the spacing between the two patterns 804A, 804B can be used to indicate the amount of time that has passed between the monitoring periods.

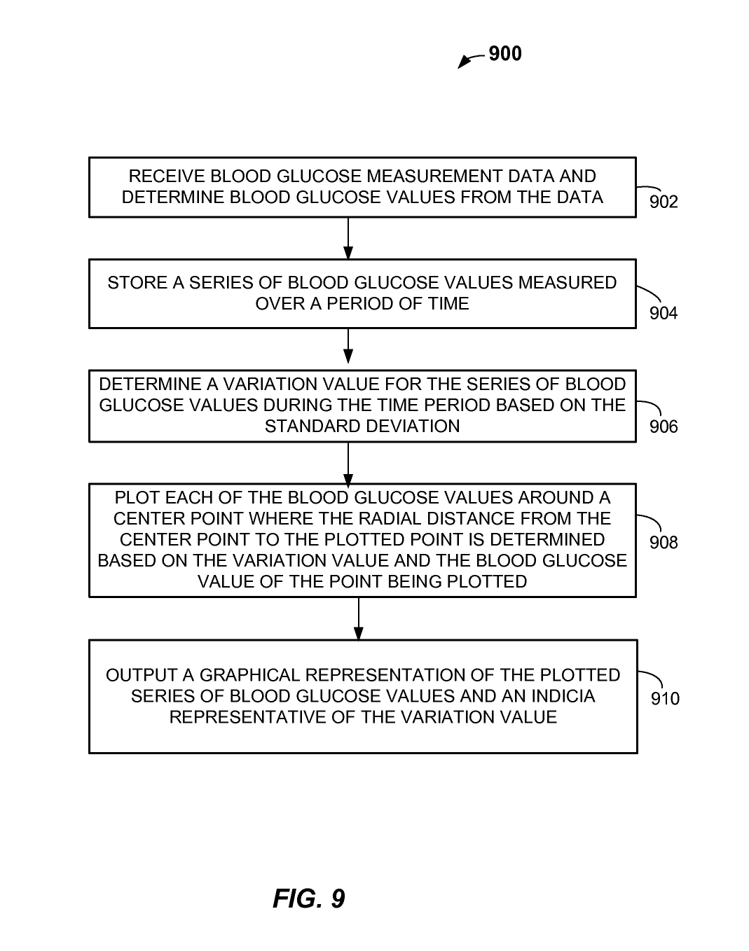

Turing now to FIG. 9, an example method 900 of concurrently displaying a graphic representation of glycemic variation and average BG is depicted in the form of a flowchart. According to embodiments of the invention, blood glucose measurement data is received (or measured) in a BGM or other computing device and BG values are determined from the data (902). Next a series of BG values measured over a period of time are stored in the BGM (or other computing device) (904). A glycemic variation value for the time period is determined for the series of stored BG values based on the standard deviation (906). Each of the blood glucose values are then plotted around a center point wherein the radial distance from the center point to the plotted point is determined based on the variation value and the blood glucose value of the point being plotted (908). A graphical representation is output on a display of the BGM (or other computing device) that represents the plotted series of blood glucose values and an indicia representative of the variation value (910).

The foregoing description discloses only example embodiments of the invention. Modifications of the above-disclosed apparatus, systems and methods which fall within the scope of the invention will be readily apparent to those of ordinary skill in the art. Accordingly, while the present invention has been disclosed in connection with example embodiments, it should be understood that other embodiments may fall within the scope of the invention, as defined by the following claims.

The invention claimed is:

1. An apparatus comprising:
a processor;
a display operatively coupled to the processor; and
a memory operatively coupled to the processor and operative to store processor executable instructions adapted to:
store in the memory a series of blood glucose values measured over a period of time,
determine using the processor a variation value for the series of blood glucose values during the time period based on a standard deviation of the series of blood glucose values,
plot points, using the processor, for each of the blood glucose values around a center point where a radial distance from the center point to each point to be plotted is determined based on the variation value and a blood glucose value of the point to be plotted, and
output to the display a graphical representation of the plotted series of blood glucose values and an indicia representative of the variation value.

2. The apparatus of claim 1 wherein the instructions further include instructions to receive blood glucose measurement data and to determine blood glucose values from the data.

3. The apparatus of claim 1 wherein the instructions to plot points include instructions to scale the radial distance based on a scaling factor.

4. The apparatus of claim 1 wherein the instructions to output a graphical representation include instructions to output the graphical representation overlaid on a multi-zone target.

5. The apparatus of claim 4 wherein the instructions to output a graphical representation overlaid on a multi-zone target include instructions to output a target having different colors in different zones.

6. The apparatus of claim 4 wherein the instructions to output a graphical representation overlaid on a multi-zone target include instructions to output a target having zones with a width determined based on the standard deviation of the series of blood glucose values.

7. The apparatus of claim 1 wherein the instructions to plot points include instructions to arrange the points based on an angular position corresponding to a modal time position related to a time at which each point to be plotted was measured.

8. The apparatus of claim 1 wherein the instructions to output a graphical representation include instructions to output a splatter pattern corresponding to the plotted points.

9. A method comprising:
storing in a memory a series of blood glucose values measured over a period of time;
determining, using a processor operatively coupled to the memory, a variation value for the series of blood glucose values during the time period based on a standard deviation of the series of blood glucose values;
plotting points, using the processor, for each of the blood glucose values around a center point where a radial distance from the center point to each point to be plotted is determined based on the variation value and a blood glucose value of the point to be plotted, and
outputting to a display operatively coupled to the processor, a graphical representation of the plotted series of blood glucose values and an indicia representative of the variation value.

10. The method of claim 9 further including receiving blood glucose measurement data and determining blood glucose values from the data.

11. The method of claim 9 wherein plotting points includes scaling the radial distance based on a scaling factor.

12. The method of claim 9 wherein outputting a graphical representation includes outputting the graphical representation overlaid on a multi-zone target.

13. The method of claim 12 wherein outputting a graphical representation overlaid on a multi-zone target includes outputting a target having different colors in different zones.

14. The method of claim 12 wherein outputting a graphical representation overlaid on a multi-zone target includes outputting a target having zones with a width determined based on the standard deviation of the series of blood glucose values.

15. The method of claim 9 wherein plotting points includes arranging the points based on an angular position corresponding to a modal time position related to a time at which each point to be plotted was measured.

16. The method of claim 9 wherein outputting a graphical representation includes outputting a splatter pattern corresponding to the plotted points.

17. A system comprising:
a processor;
a port operatively coupled to the processor and adapted to receive a blood glucose test strip;
a display operatively coupled to the processor; and
a memory operatively coupled to the processor and operative to store processor executable instructions adapted to:
measure blood glucose levels in blood samples applied to test strips received in the port and to determine blood glucose values from the measurements,
store in the memory at least two series of blood glucose values measured over at least two different periods of time,
determine using the processor, a variation value for each of the series of blood glucose values during the respective time periods based on a standard deviation of each of the series of blood glucose values,
plot points using the processor, for a first series for each of the blood glucose values around a first center point where a radial distance from the first center point to each point of the first series to be plotted is determined based on the variation value of the first series and a blood glucose value of the point to be plotted,
plot points using the processor for a second series for each of the blood glucose values around a second center point where a radial distance from the second center point to each point of the second series to be plotted is determined based on the variation value of the second series and a blood glucose value of the point to be plotted, and
output to the display graphical representations of the first and second plotted series of blood glucose values and an indicia representative of the variation values of the first and second series.

18. The system of claim 17 wherein the instructions to output graphical representations include instructions to output the graphical representations wherein each are overlaid on a different multi-zone target.

19. The system of claim 17 wherein the instructions to output graphical representations include instructions to output the graphical representations side-by-side including connecting lines that illustrate relative sizes of the graphical representations.

20. The system of claim 17 wherein the instructions to output graphical representations include instructions to output the graphical representations scaled to emphasize the relative sizes of the graphical representations.
